(19) United States
(12) Patent Application Publication
(10) Pub. No.: US 2026/0252129 A1
(43) Pub. Date: Aug. 27, 2026

(54) METHOD FOR OPERATING A HEAT EXCHANGER UNIT FOR CHANGING THE PHYSICAL STATE OF MATTER OF AN AQUEOUS FORMULATION

(71) Applicant: LONZA LTD, Visp (CH)

(72) Inventors: Philippe JEANRICHARD, Basel (CH); Patrick-Adrien HERNAIZ, Basel (CH); Christos THEODOROU, Basel (CH); Peter YAO, Basel (CH); Arundhati CHATTOPADHYAY, Basel (CH); Michael ADLER, Basel (CH)

(21) Appl. No.: 18/879,276

(22) PCT Filed: Jun. 29, 2023
(86) PCT No.: PCT/EP2023/067789

§ 371 Date: Dec. 27, 2024

(30) Foreign Application Priority Data
Jun. 30, 2022 (EP) 22182434.5
Mar. 3, 2023 (EP) 23160053.7
Jun. 6, 2023 (EP) 23177734.3

(51) Int. Cl. G05D 23/19

(52) U.S. Cl. CPC G05D 23/1904; G01K 3/04; G05B 19/0415; G01K 7/425

(57) ABSTRACT

Applicant: Lonza Ltd Title: Method for operating a heat exchanger unit for changing the physical state of matter of an aqueous formulation Our ref.: LZA27238PCT Date: Jun. 29, 2023 Page 43 of 43 Abstract Method for operating a heat exchanger unit for freezing or thawing an aqueous formulation comprising the steps of: segmenting a target temperature course into two distinct phases, wherein at least one phase refers to a change in physical state of an aqueous formulation; determining a control parameter variation, based on which operation of the heat exchanger unit is to be controlled 5 during an operating cycle; performing a test run operating cycle by operating the heat exchange unit based on the determined control parameter variation and by measuring an aqueous formulation temperature course; segmenting the measured temperature course into two distinct phases, wherein at least one phase refers to a change in physical state of matter; adapting the determined control parameter variation based on a comparison of the segmented measured substance 10 temperature course with the segmented target temperature course; and operating the heat exchanger unit based on the adapted control parameter variation.

### TECHNICAL FIELD

The present invention refers to a method and system for operating a heat exchanger unit for changing the physical state of matter of an aqueous formulation, in particular for freezing and/or thawing an aqueous formulation. Further, the present invention refers to a computer program comprising instructions which, when executed by a computer, causes the computer to perform such a method.

### TECHNOLOGICAL BACKGROUND

For properly and effectively processing drug products, e.g. in drug product manufacturing procedures, predefined storage, transportation and processing requirements may need to be strictly followed. This may be due to labile nature of drug products and may particularly apply for aqueous formulations comprising proteins.

Depending on the storage temperature, aqueous formulations of proteins may undergo various changes, in particular chemical or biochemical changes, such as enduring microbial growth, foaming upon transportation, forming aggregations, degrading overtime, etc. When using such aqueous protein solutions in pharmaceutical productions, these changes may affect the properties of a drug product to be manufactured, such as the stability of the drug product. A process of freezing and thereafter thawing protein solutions or other aqueous formulations is hence commonly used in the pharmaceutical industry in order to prevent undesired and unintended changes.

However, the process steps of freezing and thawing aqueous formulations, in particular aqueous formulations comprising proteins, must follow strictly controlled processes since both of these steps may affect the final properties of the drug product. For example, during both of these steps, denaturation may occur due to different reasons, including: cryoconcentration and co-solutes, which may also cause pH shifts and cryopreservants crystallization; cold denaturation and unfolding/misfolding; aggregation, which may lead to immunogenicity, loss of potency, or other alterations of the required critical quality attributes.

As such, the specification, implementation and validation of a process for freezing and thawing aqueous formulations to be used in pharmaceutical productions are usually time and cost intense as they require lengthy experiments and design. For performing these process steps in pharmaceutical productions, the use of heat exchanger systems is known. However, operating and setting up such heat exchanger systems such that predefined freezing and thawing processes and requirements are met during operation, may be particularly time- and cost-intensive.

Further technical background is known from PARVIZ A SHAMLOU ET AL: “A new scaleable freeze-thaw technology for bulk protein solutions”, BIOTECHNOLOGY AND APPLIED BIOCHEMISTRY, ACADEMIC PRESS, US, vol. 46, no. 1, 23 Dec. 2010 (2010-12-23), pages 13-26.

### SUMMARY OF THE INVENTION

Thus, it is an object of the present invention to provide an improved method and system for operating a heat exchanger unit for changing the physical state of an aqueous formulation by freezing or thawing, which in particular enables to reliably and efficiently operate the heat exchanger unit in line with predefined process requirements. Further, it is an object of the present invention to provide a computer program comprising instructions which, when executed by a computer, causes the computer to perform such an improved method.

These objectives are solved by the subject matter of the independent claims. Preferred embodiments are set forth in the present specification, the Figures, and the dependent claims.

Accordingly, a method for operating a heat exchanger unit is provided for changing the physical state of matter of an aqueous formulation by freezing or thawing, the aqueous formulation being contained in a container and the heat exchanger unit acting on the aqueous formulation in the container, such that a temperature of the drug product during an operating cycle is regulated based on or in accordance with a target temperature course. The method comprises the steps of:
segmenting the target temperature course into at least two distinct phases, wherein at least one phase refers to a change in physical state of the aqueous formulation;
based on the target temperature course, determining at least one control parameter variation, based on which operation of the heat exchanger unit is to be controlled during an operating cycle;
performing a test run operating cycle comprising the sub-steps of:
   operating the heat exchange unit during the test run operating cycle based on the determined control parameter variation, and
   measuring a temperature course being indicative of the course of the aqueous formulation's temperature during the test run operating cycle.

The method further comprises the steps of:
segmenting the measured temperature course into at least two distinct phases, wherein at least one phase refers to a change in physical state of the aqueous formulation;
adapting the determined control parameter variation based on a comparison of the segmented measured temperature course with the segmented target temperature course;
and
operating the heat exchanger unit based on the adapted control parameter variation for freezing and/or thawing the aqueous formulation.

By providing a method in which temperature courses are segmented into distinct phases, wherein at least one phase refers to a change in the physical state of matter of the aqueous formulation, the proposed method may allow to efficiently and effectively determine and/or adapt the control parameter variation to reliably operate the heat exchanger in accordance with the target temperature course constituting predefined process requirements.

The proposed method may be a computer-implemented method. That is, the method steps described in the following may, at least partly, involve computers, computer networks or other programmable apparatus. In other words, at least one method step is realized by means of a program.

In particular, the method of the present invention may be implemented using a computer, i.e., by storing the method steps of the present invention in memory (e.g., non-transitory memory) and executing the stored method steps using a central processing unit (CPU). As used herein, the term ‘computer program’ or ‘program’ is intended to refer to a series of method steps stored in memory configured to be carried out by a CPU.

Further, the proposed method may be used in pharmaceutical production in which an aqueous formulation may undergo predetermined and strictly controlled freezing and/or thawing processing steps. However, the proposed method is not limited to this use and may be applied in any suitable application in which changing the physical state of matter of an aqueous formulation is to be performed in compliance with strict specifications and regulations. As such, the proposed method may be applied for setting up or re-adjusting proper operation of any heat exchanger unit intended for changing a physical state of matter of an aqueous formulation.

The term ‘physical state of matter’ (which can also be called aggregate state) as used herein means a physical state such as solid, liquid or gaseous state of matter; the method of the instant invention revolves around changing the physical state between solid and liquid (by freezing or thawing).

Preferably, the aqueous formulation to be frozen and/or thawed upon carrying out the method comprises water as the main (or even sole) component, and may further comprise buffer, cell culture medium, and/or one or more drug substances. The change of the physical state of matter in the sense of the invention essentially comprises of freezing or thawing of the water in the aqueous formulation.

In the context of the present disclosure, the term ‘aqueous formulation of a drug substance’ (also often referred to as ‘drug product’) refers to any aqueous formulation comprising a drug substance in an aqueous medium such as buffer, cell culture medium and the such. A drug substance is a pharmaceutically active substance (often referred to as Active Pharmaceutical Ingredient ‘API’).

The various types of drug substances are known to the skilled person, and span from large molecules such as proteins or peptides to small organic molecules. Especially in case of large molecules, the change of the physical state of an aqueous formulation containing such a drug substance may be particularly critical as stated herein.

The term ‘protein’ means a macromolecule composed of a sequence of amino acids. A protein may be a therapeutic protein, e.g., used in diagnosis, treatment, and/or prevention of a disease or disorder. A protein can be a native protein, that is, a protein produced by a naturally-occurring and non-recombinant cell; or it can be produced by a genetically-engineered or recombinant cell and may comprise molecules having the amino acid sequence of the native protein, or molecules having deletions from, additions to, and/or substitutions of one or more amino acids of the native sequence, or molecules having the amino acid sequence of a protein with no relation to a native protein. The term also includes amino acid polymers in which one or more amino acids are chemical analogues of a corresponding naturally-occurring amino acid polymer.

Preferred protein drug substances are antibodies, which may include monoclonal antibodies and polyclonal antibodies, whole antibodies, antibody-drug conjugates, chimeric antibodies, humanized antibodies, human antibodies or hybrid antibodies with dual or multiple antigen or epitope specificities, antibody fragments and antibody sub-fragments, e.g., Fab, Fab′, F(ab′)2, fragments and the like, including hybrid fragments of any immunoglobulin or any natural, synthetic or genetically engineered protein that acts like an antibody by binding to a specific antigen to form a complex.

Suitable aqueous media are known to the skilled artisan. A preferred aqueous media is water, or an aqueous buffer.

At least one pharmaceutically acceptable excipient may also be comprised in the aqueous formulation. Suitable excipients are known to the skilled artisan such as stabilizers, pH modifying agents etc. In some embodiments, a polysorbate such as polysorbate 20, 40, 60 or 80 may be used as stabilizing agent, which can improve stability of a protein drug in an aqueous formulation.

A preferred embodiment of an aqueous formulation used in the proposed method is an aqueous formulation comprising the drug substance. According to this embodiment, a change of physical state of matter of the aqueous formulation may refer to a change of the physical state of matter of the water comprised in the aqueous formulation comprising the drug substance, i.e. freezing and/or thawing of water in the aqueous composition comprising the drug substance.

A set forth above, the method is intended and applied for controlling operation of the heat exchanger unit. The heat exchanger unit is configured for changing the physical state of matter of an aqueous formulation by freezing and/or thawing the aqueous formulation, specifically during an operating cycle. In the context of the present disclosure, the term ‘operating cycle’ refers to a time period of a predefined length during which the heat exchanger unit is operated to control heat transfer to and/or from the aqueous formulation and during which at least one change of the physical state of the aqueous formulation occurs. In other words, the heat exchanger unit may be configured for freezing and/or thawing the aqueous formulation during an operating cycle. For example, the heat exchanger unit may be operated so as to, at first, freeze and, thereafter, thaw the aqueous formulation during an operating cycle, or vice versa. Accordingly, the heat exchanger unit may also be referred to as a cooling unit, a freezing unit, a thawing unit or a freezing-thawing unit.

In general, the heat exchanger unit may be configured to control or manipulate heat transfer, in particular a heat flow, to or from the aqueous formulation. For doing so, the heat exchanger unit may comprise a controllable heat transfer component, which may also be referred to as a cooling component. Accordingly, the heat exchanger unit may be configured to control heat transfer between the heat transfer component and the aqueous formulation during the operating cycle based on the determined control parameter variation. The heat transfer component may be configured to manipulate the amount and direction of heat flow to or from the aqueous formulation. Specifically, the heat transfer component may be configured to transfer heat to or from the aqueous formulation by means of at least one of thermal conduction, thermal convection and thermal radiation. For example, the heat transfer component may comprise heat transfer plates which may be flown through by a heat transfer medium (also referred to as “cooling medium”), a flow rate and/or temperature of which is controllable by the heat exchanger unit during the operating cycle.

The heat exchanger unit may be configured to receive or accommodate the aqueous formulation to be processed. For doing so, the heat exchanger unit may comprise a receiving space for accommodating the aqueous formulation during the operating cycle. The heat transfer component may at least partly delimit the receiving space. In a received state, in which the aqueous formulation is received in the receiving space of the heat exchanger unit, a heat-conducting connection may be provided between the heat transfer component and the aqueous formulation. Specifically, a surface of the heat transfer component may constitute a surface of the receiving space, in particular an inner surface of the receiving space. The receiving space may constitute a reservoir for the aqueous formulation. According to one configuration, in the received state of the aqueous formulation, the aqueous formulation may be in direct contact or thermal contact with the surface of the receiving space.

Alternatively, the heat exchanger unit may be configured to receive or store the aqueous formulation in a container, such as a bag, a bottle, etc. In the context of the present disclosure, the term ‘container’ refers to any component configured for accommodating or storing the aqueous formulation. The container may be interchangeably received in the heat exchanger unit, in particular in the receiving space. That is, a container received in the heat exchanger unit may be replaced by another container which may be structurally identical or different, in particular which may have a different size or volume. For example, the container may have a volume in the range of 10 l to 0.25 l, but may also have a volume greater than 10 l or smaller than 0.25 l. For example, the container may have a volume of 9 l or 5 l or 0.4 l. The container may be made of plastic, such as polyethylene and/or a copolymer, in particular ethylene-vinyl alcohol copolymer. Further, the container may be intended for a single use only, e.g. only for a single operating cycle of the heat exchanger unit. Still further, the container may be intended for being used across multiple unit operations and multiple actions or cycles within a given unit operation. The container may at least partly be in direct contact or in thermal contact with the surface of the receiving space, in particular with a surface of the heat transfer component, thereby allowing to transfer heat via thermal conduction and/or thermal convection. For allowing to properly arrange the container within the receiving space, at least one insert component may be placed within the receiving space together with the container. In this way, it may be ensured that containers of varying sizes and volumes may be properly arranged within the receiving space.

As set forth above, for changing a physical state of matter, the heat exchanger unit is configured to control or manipulate a heat flow to or from the aqueous formulation, in particular to or from the container. For doing so, the heat exchanger unit may comprise a control unit configured to control operation of the heat exchanger unit, in particular of the heat transfer component, based on at least one control parameter. In the context of the present disclosure, the term ‘control parameter’ refers to a parameter, based on which operation, in particular process variables, of the heat exchanger unit, in particular of the heat transfer component, is controlled. Thus, upon changing a control parameter, operation or process variables of the heat exchanger unit is/are affected. In other words, upon changing a control parameter during operation of the heat exchanger unit, heat transfer to or from the aqueous formulation may be manipulated. The control parameter may be indicative of a process variable of the heat exchanger unit to be set. For example, the control parameter may be indicative of or may refer to a temperature of the heat transfer component to be set during operation of the heat exchanger unit. More specifically, the control parameter may be indicative of or may refer to a temperature of the cooling medium flowing through the heat transfer component. Preferably, the control parameter is indicative of a predetermined temperature of the heat transfer medium and may be a temperature set point of the cooling medium. Alternatively or additionally, the control parameter may be indicative of or may refer to a flow rate of the cooling medium flowing through the heat transfer component. In other words, the control parameter variation, i.e. the variation of the control parameter, effects a desired change of a control variable of the heat exchanger unit, in particular a change of temperature and/or flow rate of the heat transfer medium flowing through the heat transfer component. In other words, the control parameter variation, i.e. the variation of the control parameter, effects a desired change of a control variable of the heat exchanger unit, in particular a change of temperature and/or flow rate of the heat transfer medium flowing through the heat transfer component.

In this context, the term ‘control parameter variation’ refers to a variation or course of at least one control parameter of the heat exchanger unit during the operating cycle. Thus, the control parameter variation indicates how at least one process variable of the heat exchanger unit is to be set and changed overtime, i.e. during the operating cycle. As such, the control parameter variation associates to each one of different points in time during the operating cycle one value of the control parameter. Accordingly, the control parameter variation may be provided or expressed in the form of a function or curve that assigns to or defines for a set of points in time or each point in time during the operating cycle exactly one value of the control parameter. For example, the at least one control parameter variation may be indicative of a course of a temperature of the heat transfer component during the operating cycle. Alternatively or additionally, the at least one control parameter variation may be indicative of a course of a temperature or a flow rate of the heat transfer medium during the operating cycle. Preferably, the control parameter variation is indicative of or is a course of a predetermined temperature, i.e. a course of a temperature set point, of the heat transfer medium.

Specifically, the control parameter variation may define a change of the control parameter of the heat exchanger unit during the phase referring to the change in the physical state of the aqueous formulation, i.e. during freezing and/or thawing phase, in particular during a freezing phase, and/or during at least one of a cooling and a worming phase. The cooling or warming phase may refer to a phase during which the temperature of the aqueous formulation is decreased or increased. More specifically, the control parameter variation may define or be indicative of a change of the temperature of the heat transfer component during the phase referring to the change in the physical state of the aqueous formulation, i.e. during freezing and/or thawing phase, in particular during the freezing phase and/or during at least one of a cooling phase and a warming phase. Further, the control parameter variation may define or be indicative of a change of the temperature and/or the flow rate of the cooling medium flowing through the heat exchanger unit during the phase referring to the change in the physical state of the aqueous formulation, i.e. during freezing and/or thawing phase, in particular during the freezing phase and/or during at least one of a cooling phase and a warming phase.

For operating the heat exchanger unit based on the control parameter or the control parameter variation, the control unit may apply a feedback control, such as a closed-loop control, for ensuring that process variables of the heat exchanger unit are properly set in accordance with the control parameter. For example, in case the control parameter is indicative or refers to the temperature of the heat transfer component, the heat exchanger unit may be equipped with a temperature sensor configured for measuring a temperature of the heat transfer component and for sending a feedback signal to the control unit being indicative of the measured temperature.

A person skilled in the art understands that the method may be performed by using different types of heat exchanger units and that the present invention is not limited to a specific configuration of the heat exchanger unit. The structure and function of such heat exchanger units and their components are well known to a person skilled in the art and are thus not further specified. Rather, characteristics of the method for operating such a heat exchanger unit for changing the physical state of matter of an aqueous formulation which are interlinked with the present invention are addressed in the following.

The proposed method is intended to operate the heat exchanger unit such that the aqueous formulation is subjected to temperature changes during the operating cycle that may follow or mimic the target temperature course. In the context of the present disclosure, the term ‘target temperature course’ thus refers to an intended or predefined course of a temperature to prevail in the aqueous formulation during the operating cycle of the heat exchanger unit.

The target temperature course may relate to a temperature course of an aqueous formulation processed by another heat exchanger unit. The other heat exchanger unit may be a reference heat exchanger unit, the operation of which is to be imitated by the heat exchanger unit. In other words, the proposed method may be intended for operating the heat exchanger unit such that a temperature course of an aqueous formulation to be processed mimics or corresponds to a temperature course the aqueous formulation undergoes when being processed by the reference heat exchanger unit. The reference heat exchanger unit may have different aqueous formulation processing properties compared to the heat exchanger unit to be controlled by the proposed method. Specifically, the heat exchanger unit to be controlled by the method may be a scaled model, in particular a scaled down model of the reference heat exchanger unit. For example, the reference heat exchanger unit may have a different, in particular a greater aqueous formulation processing capacity compared to the heat exchanger unit to be controlled by the method. In other words, a maximum amount of the aqueous formulation to be processed during an operating cycle may differ among the heat exchanger unit and the reference heat exchanger unit.

Accordingly, the target temperature course may refer or correspond to a temperature variation the aqueous formulation is subjected to upon being processed by the reference heat exchanger unit for changing a physical state. For determining the target temperature course, the method may further comprise a step of operating the reference heat exchanger unit for changing a physical state of the aqueous formulation during an operating cycle and a step of measuring a temperature course of the aqueous formulation upon being processed in the reference heat exchanger unit during the operating cycle. As such, the target temperature course may correspond to the temperature course of the aqueous formulation when being processed in or by the reference heat exchanger.

The method further comprises the step of segmenting the target temperature course into at least two distinct phases. In the context of the present disclosure, the term ‘distinct phases’ may refer to non-overlapping phases or segments of the target temperature course, but which are adjacent to each other without a gap in between. By segmenting the target temperature course, different phases of the target temperature course are identified and defined.

The distinct phases of the target temperature course may refer to one of a temperature varying phase, a temperature holding phase or a changing physical state phase. The temperature varying phase may refer to a time period in the operating cycle during which the temperature of the aqueous formulation is to be varied over time, i.e. without changing the aqueous formulation's physical state. The temperature holding phase may refer to a time period in the operating cycle during which the temperature of the aqueous formulation is to be held on a constant or substantially constant level without changing the aqueous formulation's physical state. The changing physical state phase may refer to a time period in the operating cycle, during which a physical state of the aqueous formulation is to be changed, i.e. without varying or without substantially varying the temperature of the aqueous formulation.

When during a changing of the physical state phase the aqueous formulation is frozen, then such a changing of the physical state phase may be called a freezing phase.

When during a changing of the physical state phase the aqueous formulation is thawed, then such a changing physical state phase may be called a thawing phase.

The target temperature course may comprise a plurality of different and distinct temperature variation phases which may differ from one another, e.g. in view of a temperature change rate and/or a temperature level. Alternatively or additionally, the target temperature course may comprise a plurality of different and distinct temperature holding phases which may differ from one another, e.g. in view of a temperature level. Alternatively or additionally, the target temperature course may comprise a plurality of different changing physical state phases.

According to one configuration, the target temperature course may comprise at least two of the following eight phases:
a first phase referring to a temperature variation phase during which the aqueous formulation is to be cooled until freezing, i.e. until reaching the freezing point of the aqueous formulation,
a second phase referring to a change of the physical state during which the aqueous formulation is frozen, a freezing phase;
a third phase referring to a further temperature variation phase during which the aqueous formulation is to be cooled until reaching a predefined temperature being lower than the freezing point;
a fourth phase referring to a temperature holding phase during which the aqueous formulation is held at the predefined temperature;
a fifth phase referring to a further temperature variation phase during which the aqueous formulation is to be warmed up until reaching a thawing point of the aqueous formulation;
a sixth phase referring to a further change of physical state phase during which the aqueous formulation is thawed, a thawing phase;
a seventh phase referring to a further temperature variation phase during which the aqueous formulation is to be heated until reaching a further predefined temperature being greater than the thawing point; and
an eighth phase referring to a further temperature holding phase during which the aqueous formulation is held at the further temperature;
said first and third phases may also be called a cooling phase, said fifth and seventh phases may also be called a warming phase;
with at least one of the two phases being a change of physical state of matter phase and with the at least two phases being adjacent to each other.

Specifically, the step of segmenting the target temperature course into at least two distinct phases is performed such that at least one of the segmented phases, i.e. identified during this method step, refers to a change in the physical state of the aqueous formulation, in particular to freezing or thawing the aqueous formulation. In other words, at least one of the phases identified in the step of segmenting the target temperature course refers to a changing the physical state phase as described above, such as a freezing phase or a thawing phase. Preferably, during this phase, a change in the physical state of the aqueous formulation is to be commenced and completed. The step of segmenting the target temperature course may be performed to identify at least one of a freezing phase, during which freezing of the aqueous formulation aqueous formulation is to be performed, and a thawing phase, during which thawing of the aqueous formulation is to be performed. Further, the step of segmenting the target temperature course may be performed to segment the target temperature course into at least two of the above described first to eighth phases adjacent to each other, with one of the at least two of the above described first to eighth phases being the thawing phase or the freezing phase, and without a gap in between the phases. According to one configuration, the target temperature course may be segmented into the above described first to eighth phases which successively follow one another.

The step of segmenting the target temperature course may include identifying endpoints, which can also be referred to as cutoff points or limiting points or starting and ending points, of the respective phases, i.e. points at which the different phases start and end. Each identified phase may be defined by its starting point and its ending point. In other words, the step of segmenting the target temperature course may include segmenting the target temperature course into phases by identifying endpoints of the phases.

The step of segmenting the target temperature course may be realized by means of a computer program. Specifically, this step may be performed in an automated manner. For doing so, a computer program, e.g. running on the control unit of the heat exchanger unit, may be configured to analyze the target temperature course based on a freezing and thawing point of the aqueous formulation to identify phases during which the aqueous formulation is to be held on a temperature level corresponding to the freezing and/or thawing point, these phases being changing the physical state of matter phases. Further, the computer program may analyze the target temperature course so as to identify phases during which the temperature of the aqueous formulation is to be varied overtime, these phases may be temperature variation phases. Still further, the program may analyze the target temperature course to identify phases during which the temperature of the aqueous formulation is to be held on a temperature level being different from the freezing and thawing point of the aqueous formulation, these phases being temperature holding phases.

Alternatively or additionally, the segmenting of the target temperature course may be obtained from a database. Alternatively or additionally, the step of segmenting the target temperature course may be performed based on a user interaction or a user input. For doing so, for example, a user may provide an input to the computer program which is indicative of endpoints of the different phases. Based on this input, the computer program may identify and define the different phases of the target temperature course.

Alternatively or additionally, the step of segmenting the target temperature course may include processing the segmented target temperature course, i.e. the identified different phases thereof. Specifically, the step of segmenting the target temperature course may include adapting the phases of the target temperature course by applying a regression analysis method. Specifically, the step of segmenting the target temperature course may include adapting the phases of the target temperature course by applying linear regression. Alternatively or additionally, the step of segmenting the target temperature course may include adapting the phases of the target temperature course by applying the method of least squares or any other suitable regression analysis method. By doing so, the course of the target temperature course may be piecewise, i.e. phase-wise, adapted. For applying linear regression, at least one of the identified phases of the target temperature course, in particular each one of the phases, may be adapted such that the course of the target temperature within the distinct phases complies with the following equation:

$$Y = a + b * X \qquad ( 1 )$$

wherein Y refers to a value of the temperature, a refers to a coefficient or intercept, b refers to a slope, and X refers to a point in time. Accordingly, for each phase of the target temperature course, the coefficient a and the slope b may be determined. By applying linear regression, computing effort for performing the method may be decreased. In particular, by applying linear regression to the target temperature course Tt(f), the target temperature course may be segmented into phases in a manner that reduces the computational power needed in order to achieve phase segmentation, thus improving the speed and efficiency of deriving the segmented target temperature course.

The method further comprises the step of determining at least one control parameter variation. Such a control parameter variation refers to a variation of the above described control parameter over time, based on which the heat exchanger unit is to be operated during the operating cycle. The step of determining the control parameter variation is performed based on the target temperature course, in particular based on the segmented target temperature course, i.e. the target temperature course after being processed in the step of segmenting the target temperature course. In a further development, the step of determining the control parameter variation may be based on or be performed as a function of properties of the aqueous formulation, in particular based on physical or chemical properties of the aqueous formulation. For example, the step of determining the control parameter variation may be performed as a function of a volume of the aqueous formulation to be processed by the heat exchanger unit, in particular as a function of a volume of the container accommodating the aqueous formulation.

Alternatively or additionally, the step of determining the control parameter variation may be performed as a function of a type of the aqueous formulation or as a function of a heat conductivity of the aqueous formulation. Specifically, the step of determining the control parameter variation may be performed based on thermodynamic properties of the aqueous formulation. As such, the control parameter variation may be determined based on a freezing or thawing point of the aqueous formulation. For example, in case the aqueous formulation is an aqueous formulation containing a drug substance, the step of determining the control parameter variation may be performed based on or considering the freezing and/or thawing point of water. Specifically, the step may be performed such that, during a freezing phase, the temperature set point of the heat transfer medium is set below the freezing point of water and, during a thawing phase, the temperature set point of the heat transfer medium is set above the thawing point of water.

The step of determining the control parameter variation may further comprise a sub-step of identifying a freezing phase in the target temperature course, in particular in the segmented target temperature course, i.e. the target temperature course provided as an output of the step of segmenting the target temperature course. Further, a time period of the operating cycle may be determined which corresponds to the identified freezing phase, i.e. during which the aqueous formulation is to be subjected to freezing during the operating cycle.

In the context of the present invention, it has been found that a temperature shift during freezing of an aqueous formulation may occur, in particular when a volume of the aqueous formulation is relatively small. This temperature shift may be caused by super-cooling effects. Specifically, the temperature shift may occur due to a sudden release of energy which may be induced by sudden nucleation of the aqueous formulation. For preventing such sudden temperature shifts, i.e. induced by sudden nucleation, during freezing, the step of determining the control parameter variation may further comprise a sub-step of implementing a rise or decent, also referred to as a spike, in the control parameter variation in the time period corresponding to and/or preceding the freezing phase. The rise or decent in the control parameter variation may be defined to induce an increase, in particular a temporal increase, of the heat exchanger unit's cooling performance. Specifically, the rise or decent in the control parameter variation may be defined to decrease, temporarily decrease, the heat transfer component's temperature, before and/or at the beginning of the freezing phase.

In the following, an exemplary embodiment is described, in which the rise or decent in the control parameter variation is implemented. In this exemplary embodiment, the control parameter may be a temperature set point of the cooling medium flowing through the heat transfer component. Specifically, in a phase, e.g. temperature decrease phase, preceding the freezing phase, the control parameter, i.e. the temperature set point, may be decreased to a nucleation spike temperature. The nucleation spike temperature may be in the range of −22.5° C. to −37.5° C., preferably between −25° C. to −35° C., for example −30° C. Then, alternatively or additionally, the control parameter may be held at the nucleation spike temperature for a predetermined period of time, which may also be referred to as nucleation spike hold time. Specifically, the nucleation spike hold time may be in the range of 5% to 50% of the duration of the phase, e.g. the temperature decrease phase, preceding the freezing phase. Then, alternatively or additionally, the control parameter is raised from the nucleation spike temperature to a freezing temperature. This may be performed during a time period corresponding to the beginning of the determined freezing phase. Specifically, the step of raising the control parameter from the nucleation spike temperature to the freezing temperature may be performed during a time period which may have a duration in the range of 1% to 20% of the duration of the freezing phase. The freezing temperature may be in the range of −5° C. to −20° C., preferably in the range of −10° C. to −17.5° C., more preferably in the range of −12.5° C. to −17.5° C. Alternatively or additionally, thereafter, i.e. after the control parameter reaches the freezing temperature, the control parameter is kept at this freezing temperature, in particular until the aqueous formulation is frozen.

As such, in the exemplary embodiment, in the phase preceding the freezing phase, e.g. a temperature decrease phase, the control parameter, in a first sub-phase, is decreased until reaching the nucleation spike temperature and thereafter, in a second sub-phase, is kept at this temperature. Then, the control parameter, in a first sub-phase of the freezing phase, is raised to the freezing phase and thereafter, in a second sub-phase of the freezing phase, is kept at the freezing temperature.

In a further development, an operator may set at least one of the nucleation spike temperature, the nucleation spike hold time, the freezing temperature and the thawing temperature.

In a further development, characteristics of the rise or descent, such as a form thereof, in particular a slope and height thereof, to be implemented in the control parameter variation may be set based on properties, in particular physical or chemical properties of the aqueous formulation, such as a type or volume of the aqueous formulation.

The method further comprises the step of performing a test run operating cycle. In the context of the present disclosure the term ‘test run operating cycle’ may refer to a test run of the heat exchanger unit, during which proper operation of the heat exchanger unit may not be ensured and which is used to properly set up or readjust operation of the heat exchanger unit. Specifically, this step comprises the sub-step of operating the heat exchange unit during the test run operating cycle based on the determined control parameter variation. As such, the determined control parameter variation serves as a set of preliminary control parameters. In a further sub-step, a substance temperature course is measured during the test run operating cycle. In the context of the present disclosure, the term ‘substance temperature course’ refers to a course of a temperature prevailing in the aqueous formulation during operation of the heat exchanger unit. In other words, the substance temperature course is indicative of the course of the aqueous formulation's temperature during the test run operating cycle. For measuring and monitoring the aqueous formulation's temperature, the heat exchanger unit may further comprise a temperature sensor configured to measure a temperature prevailing in the aqueous formulation during operation of the heat exchanger unit. Preferably, the temperature sensor may be configured to measure the aqueous formulation's temperature at a center or substantially at a center of the aqueous substance. E.g., during the course of the freezing of the aqueous formulation, the freezing will start at the outer border of the aqueous formulation that touches the wall of the container in which the aqueous formulation is contained, and will progress into the center of the aqueous formulation until the whole volume of the aqueous formulation is frozen and solid. It may happen that a drug substance or any other dissolved component actually starts accumulating in the center of the volume of the aqueous substance due to the known decrease of the freezing point of water by a dissolved substance. Placing the temperature sensor at a center or substantially at a center of the aqueous substance takes a possible accumulation of a component of the aqueous formulation during the course of freezing into consideration. Specifically, the temperature sensor may be configured to measure the aqueous formulation's temperature at a center of the container accommodating the aqueous formulation. According to one embodiment, the temperature of the aqueous formulation is detected by a sensor which is positioned in the aqueous formulation, in particular in the container.

The method further comprises the step of segmenting the measured substance temperature course into at least two distinct phases, wherein at least one phase refers to a change in physical state of the aqueous formulation. This step may be performed corresponding to the step of segmenting the target temperature course as described above. Accordingly, all features described in connection with the step of segmenting the target temperature course may also apply and are thus disclosed for the step of segmenting the measured substance temperature course. Specifically, the step of segmenting the measured temperature course may be performed to identify at least one of a freezing phase, during which freezing of the aqueous formulation has been performed, and a thawing phase, during which thawing of the aqueous formulation has been performed. Alternatively or additionally, the step of segmenting the measured substance temperature course may be performed so as to identify different phases which, in view of their number and type, correspond to those phases identified as a result of the step of segmenting the target temperature course. Further, the step of segmenting the measured substance temperature course may include segmenting the measured substance temperature course into phases by identifying endpoints of the phases. Alternatively or additionally, the step of segmenting the measured substance temperature course may include adapting the phases of the measured temperature course by applying linear regression.

The method further comprises the step of adapting the determined control parameter variation based on a comparison of the segmented measured substance temperature course with the segmented target temperature course. Specifically, the comparison of the segmented measured substance temperature course with the segmented target temperature course may be performed phase-wise. That is, corresponding phases among the segmented measured substance temperature course and the segmented target temperature course may be identified and compared. In the context of the present disclosure, the term ‘corresponding phases’ may refer to a phase in the target temperature course and a phase in the measured temperature course during which the course of the temperature in the target temperature course and the course of the temperature in the measured substance temperature course both refer to either a temperature rising phase, during which the temperature increases, or a temperature falling phase, during which the temperature decreases, or a temperature holding phase, during which the temperature is kept on a constant temperature level being different from a freezing or thawing point, or a freezing phase or a thawing phase. Further, the phase in the target temperature course and the phase in the measured temperature course constituting corresponding phases may occur at overlapping or at substantially the same or at close time periods during the operating cycle.

Specifically, the step of adapting the control parameter variation may comprise the sub-steps of: identifying corresponding phases among the segmented measured substance temperature course and the segmented target temperature course; determining durations of the corresponding phases and/or determining average temperatures of the corresponding phases; and based on comparison of the determined durations and/or average temperatures of the corresponding phases, adapting the determined control parameter variation.

Alternatively or additionally, the step of adapting the control parameter variation may comprise the sub-steps of: identifying corresponding freezing phases or corresponding thawing phases among the segmented measured substance temperature course and the segmented target temperature course; determining durations and average temperatures of the corresponding freezing phases or the corresponding thawing phases; and determining an average value of the control parameter variation in a time period corresponding to the freezing phase or thawing phase in the segmented measured temperature course; and adapting the control parameter variation as a function of the determined durations of the corresponding freezing phases or the corresponding thawing phases, the determined average temperatures of the corresponding freezing phases or corresponding thawing phases, and the determined average value of the control parameter variation.

Further, a data processing system or apparatus is provided which comprises means for carrying out the steps of the method as described above. In particular, the invention relates to a data processing system for performing the method according to the invention. The data processing system may comprise a control unit. In one embodiment, the data processing system may comprise a CPU and memory. In another embodiment, the data processing system may further comprise a central server for running at least part of the software remote from control unit. The data processing system may be or comprise a computer. The computer may be any computing unit, such as a control unit, a personal computer or an application specific computer, which in particular is suitable or configured to interact with a heat exchanger unit. In general, such a data processing system comprises, among other things, at least a central processing unit (CPU) for executing digital instructions, and memory for storing digital instructions (e.g., computer programs) to be executed by the CPU, as will be apparent to one of skill in the art in view of the present disclosure. Alternatively or additionally, the data processing system may comprise sensors and/or actuators, e.g., sensors and/or actuators carried by or connected to the heat exchanger of the present invention, as will hereinafter be discussed in further detail. Alternatively or additionally, the data processing system may comprise interfaces to communicate with sensors and/or actuators.

The invention also relates to a computer program for performing the method according to the invention. In particular, a computer program is provided which comprises instructions which, when the program is executed by a computer, cause the computer to carry out the steps of the method as described above. The computer may be any computing unit, such as a control unit, a personal computer or an application specific computer, which in particular is suitable or configured to interact with a heat exchanger unit. As noted above, and as will be appreciated by one of ordinary skill in the art, the computer generally comprises, among other things, a CPU and memory for storing the computer program that is to be used to control the heat exchanger unit.

Still further, a computer-readable medium is provided which has stored thereon the above mentioned computer program.

Still further, a system is provided which comprises a heat exchanger unit and a control unit for operating the heat exchanger unit for changing the physical state of an aqueous formulation such that a temperature of the aqueous formulation is regulated during an operating cycle based on a target temperature course, wherein the control unit is configured to carry out the steps of the method as described above. It will be appreciated that, if desired, the control unit may comprise the aforementioned computer for carrying out the method steps as described above.

### BRIEF DESCRIPTION OF THE DRAWINGS

The present disclosure will be more readily appreciated by reference to the following detailed description when being considered in connection with the accompanying drawings in which:

FIG. 1 is a schematic view of an embodiment of a heat exchanger system;

FIG. 1a is a schematic view of an embodiment of a control unit;

FIG. 2 shows a flow diagram depicting one embodiment of a method for operating a heat exchanger unit of the heat exchanger system depicted in FIG. 1;

FIG. 3 shows a diagram illustrating a target temperature course;

FIGS. 4 and 5 show diagrams illustrating a segmented target temperature course; and

FIG. 6 shows a diagram illustrating a control parameter variation.

### DETAILED DESCRIPTION OF PREFERRED EMBODIMENTS

In the following, the invention will be explained in more detail with reference to the accompanying Figures. In the Figures, like elements are denoted by identical reference numerals and repeated description thereof may be omitted in order to avoid redundancies.

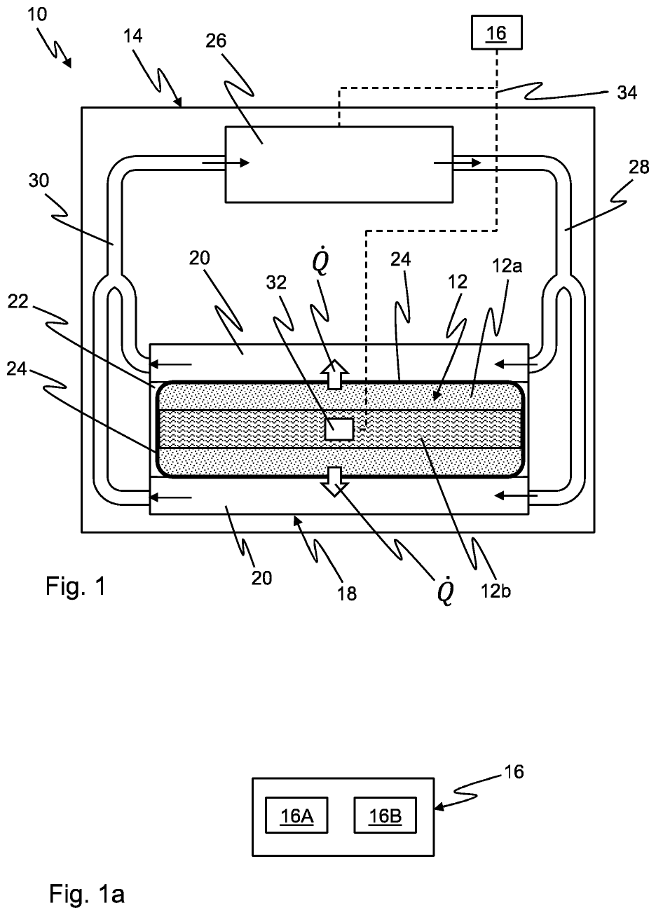

FIG. 1 shows a heat exchanger system 10, also referred to as ‘the system’ in the following, which is intended and configured to subject an aqueous formulation 12, to a predetermined and strictly controlled freezing and thawing procedure.

The shown system 10 may be used in the field of pharmaceutical production, but is not limited to this application. The system 10 comprises a heat exchanger unit 14 configured for freezing and thawing the aqueous formulation 12 and a control unit 16 configured for controlling operation of the heat exchanger unit 14.

One embodiment of the control unit 16 is shown in FIG. 1a, where the control unit comprises a central processing unit CPU 16A and a memory 16B.

The heat exchanger unit 14 may comprise a heat transfer component 18 having two heat transfer plates 20 which partly delimit, i.e. on opposing sites, a receiving space 22 for accommodating the aqueous formulation 12 to be processed during operation of the heat exchanger unit 14. Specifically, the receiving space 22 is configured to accommodate a container 24 in which the aqueous formulation 12 is received. The container 24 may be interchangeably received in the receiving space 22. Further, the container 24 may be made of polyethylene and/or an ethylene-vinyl alcohol copolymer and may be intended for a single use only. Specifically, as can be gathered from FIG. 1, the container 24 is received in the receiving space 22 such that it is in thermal contact with the heat transfer component 18, in particular with its heat transfer plates 20.

Further, the heat exchanger unit 14 comprises a temperature control unit 26 configured to regulate heat transfer performance of the heat transfer component 18 by controlling a temperature and flow rate of a heat transfer medium to be guided through the heat transfer plates 20. For doing so, the temperature control unit 26 is fluid-communicatively connected to the heat transfer plates 20 via a feed line 28 and a discharge line 30 to form a heat transfer medium circuit allowing to circulate the heat transfer medium successively through the temperature control unit 26 and the heat transfer component 18. The feed line 28 is configured for supplying the heat transfer medium to the heat transfer component 18 after being processed in the temperature control unit 26. The discharge line 30 is configured for recirculating the heat transfer medium to the temperature control unit 26 after being guided through the heat transfer component 18. By such a configuration, the heat transfer medium is configured to induce a heat transfer, in particular a heat flow Q as indicated in FIG. 1, between the aqueous formulation 12 and the heat transfer component 18 upon flowing through the heat transfer plates 20. Further, the heat exchanger unit 14 comprises a temperature sensor 32 configured to measure a temperature prevailing in the aqueous formulation 12, in particular prevailing in a center area of the aqueous formulation. By means of the temperature sensor 32, monitoring data being indicative of the temperature prevailing in the substance 12 over time may be recorded by a separate measuring device or the control unit 16.

In the shown configuration, the heat exchanger system 10 is depicted in a state in which the aqueous formulation 12 received in the heat exchanger unit 14 is subjected to a freezing procedure. In this state, a nucleation of the aqueous formulation 12 propagates from ends of the container 24 being in thermal contact with the heat transfer plates towards a center of the container. Accordingly, the aqueous formulation 12 is in part in a solid state 12a and in part in a liquid state 12b as depicted in FIG. 1. In this state, heat may be transferred from the aqueous formulation 12 towards the heat transfer plates and thus towards the heat transfer medium flowing therethrough as indicated by the direction of the heat flow Q in FIG. 1.

As set forth above, the control unit 16 is configured for controlling operation of the heat exchanger unit 14. In a one form of the invention, control unit 16 comprises a computer configured to perform the method steps of the present invention. More particularly, control unit 16 generally comprises a central processing unit (CPU) 16A configured to execute digital instructions and provide an output, and a memory 16B for storing digital instructions (e.g., a computer program) and/or output received from CPU 16A, as will be apparent to one of skill in the art in view of the present disclosure. In order to control operation of the heat exchanger unit 14, the control unit 16 is communicatively connected to the heat exchanger unit 14, in particular to the temperature control unit 26, via at least one data interface 34. The data interface 34 may be a wireless interface or a wired interface. Specifically, the control unit 16 is configured to send control signals via the data interface 34 to the heat exchanger unit 14, in particular to the temperature control unit 26, based on which operation of the heat exchanger unit 14 is controlled.

In the shown configuration, the control unit 16 comprises a computer, for example a personal computer or an application specific computer, provided separately from the heat exchange unit 14. As such, the control unit 16 is provided as a separate component which is communicatively and detachably connected to the heat exchanger unit 14 via the data interface 34. Alternatively, the control unit 16 may be provided as a part of the heat exchanger unit 14. That is, the control unit 16 may be an application specific computer integrated in the structural configuration of the heat exchanger unit 14.

The control unit 16 is configured to carry out a method for operating a heat exchanger unit 14 for changing the physical state of the aqueous formulation 12 such that a temperature of the aqueous formulation 12 during an operating cycle is regulated based on a target temperature course T_{T}(t). Specifically, the control unit 16 constitutes or is part of a data processing apparatus or system. Further, as noted above, the control unit may comprise (or may have access to) a computer program or computer program product or a computer-readable medium comprising or storing instructions which, when being executed by the control unit 16, cause the control unit 16 to carry out the method for operating the heat exchanger unit 14.

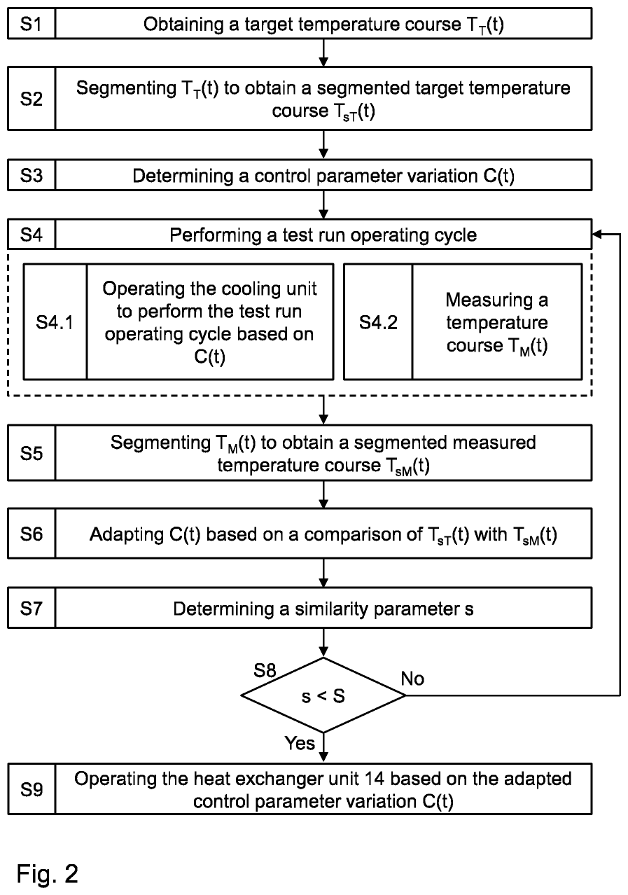

The method carried out by the control unit 16 for operating the heat exchanger unit 14 is depicted in FIG. 2 in the form of a flow diagram. In the following, the method and its individual steps carried out by the control unit 16 are specified with reference to FIGS. 2 to 6.

In a first step S1 of the method, a target temperature course T_{T}(t) is obtained, for example from a database. Specifically, the target temperature course T_{T}(t) may be provided as an input to the control unit 16. More specifically, the target temperature course T_{T}(t) may be provided in the form of a function or of a plurality of set of points which assign to points in time during the operating cycle exactly one value of a temperature. As such, the target temperature course may be provided in the form of a continuous or discontinuous function or in the form of discontinuous data points.

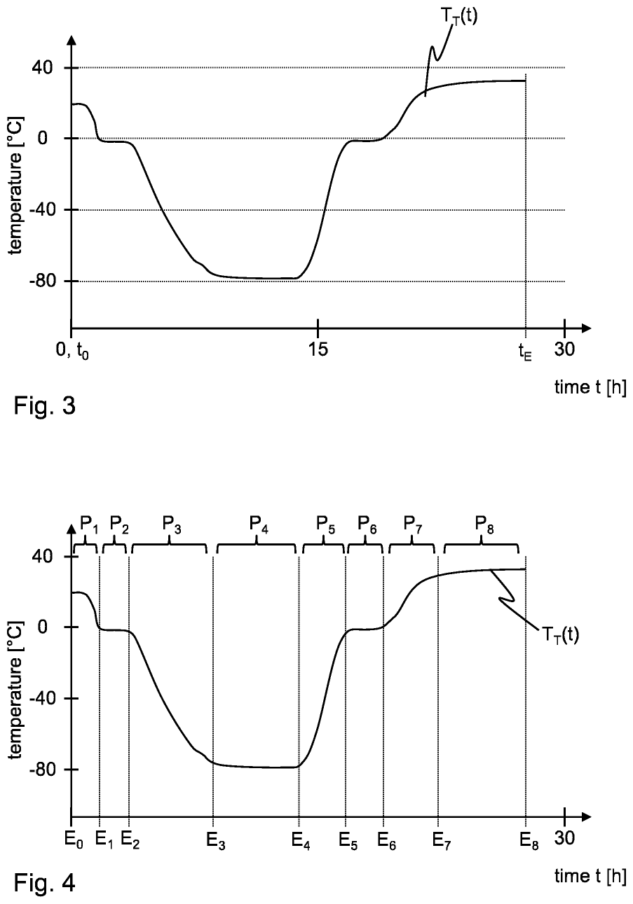

FIG. 3 depicts an exemplary target temperature course illustrated as a graph in a diagram. The ordinate of the diagram depicts a temperature, in particular a target temperature to prevail in the aqueous formulation 12. The abscissa of the diagram depicts the time, wherein to refers to a starting point of the operating cycle and t_{E} refers to an ending point of the operating cycle.

The target temperature course indicates a desired or intended variation of the aqueous formulation's temperature during an operating cycle of the heat exchanger unit 14. The target temperature course T_{T}(t) may correspond to a temperature course, the aqueous formulation is subjected to upon being processed by another heat exchanger unit, in particular a reference heat exchanger unit. The target temperature course T_{T}(t) may in particular correspond to a predetermined temperature course specific to the aqueous formulation, as derived empirically using a reference heat exchanger unit and stored in a database (e.g., a database stored in memory 16B or otherwise accessible by CPU 16A and/or control unit 16). Accordingly, upon operating the heat exchanger unit 16 in accordance with the target temperature course T_{T}(t), operation of the reference heat exchanger unit may be imitated by the heat exchanger unit 16. Specifically, the heat exchanger unit 16 may be a scaled model, in particular a scaled down model, of the reference heat exchanger unit.

In a next step S2, the target temperature course T_{T}(t) is segmented into at least two distinct phases P₁₋₈, wherein at least one phase P₂, P₆ refers to a change in physical state of the aqueous formulation 12. In the present configuration, the method may distinguish between the following different types of phases: temperature varying phases, temperature holding phases, freezing phases and thawing phases. For segmenting the target temperature course, in a first sub-step, endpoints E₀₋₈ of the different phases may be identified which refer to specific point in times during the operating cycle. This may be performed by, at first, identifying phases during which no or only small temperature variations occur. These phases may then be compared to the freezing point and thawing point of the aqueous formulation so as to decide whether these phases correspond to a temperature holding phase, a freezing phase or a thawing phase. Thereafter, temperature varying phases are identified which may be disposed between identified phases, temperature holding phases, freezing phases and thawing phases. Specifically, the step S2 of segmenting the target temperature course T_{T}(t) is performed to identify a freezing phase P₂, during which freezing of the aqueous formulation 12 is to be performed, and a thawing phase P₆, during which thawing of the aqueous formulation 12 is to be performed.

More specifically, upon subjecting the target temperature course T_{T}(t) to step S2, eight distinct phases P₁₋₈ may be determined as depicted in FIG. 4. A first phase P₁ refers to a temperature variation phase during which a temperature of the aqueous formulation 12 is to be successively decreased from 20° C. to the freezing point of about 0° C. A second phase P₂ refers to a freezing phase during which the aqueous formulation 12 is to be frozen. A third phase P₃ refers to a temperature variation phase during which the temperature of the aqueous formulation 12 is to be successively decreased from the freezing point to about −80° C. A fourth phase P₄ refers to a temperature holding phase during which the temperature of the aqueous formulation 12 is to be held at about −80° C. A fifth phase P₅ refers to a temperature variation phase during which the temperature of the aqueous formulation 12 is to be successively increased from about −80° C. to the thawing point. A sixth phase P₆ refers to a thawing phase during which the aqueous formulation 12 is to be thawed. A seventh phase P₇ refers to a temperature variation phase during which the temperature of the aqueous formulation 12 is to be successively increased from the thawing point to about 25° C. An eighth phase P₈ refers to a temperature holding phase during which the temperature of the aqueous formulation 12 is to be held at about 25° C.

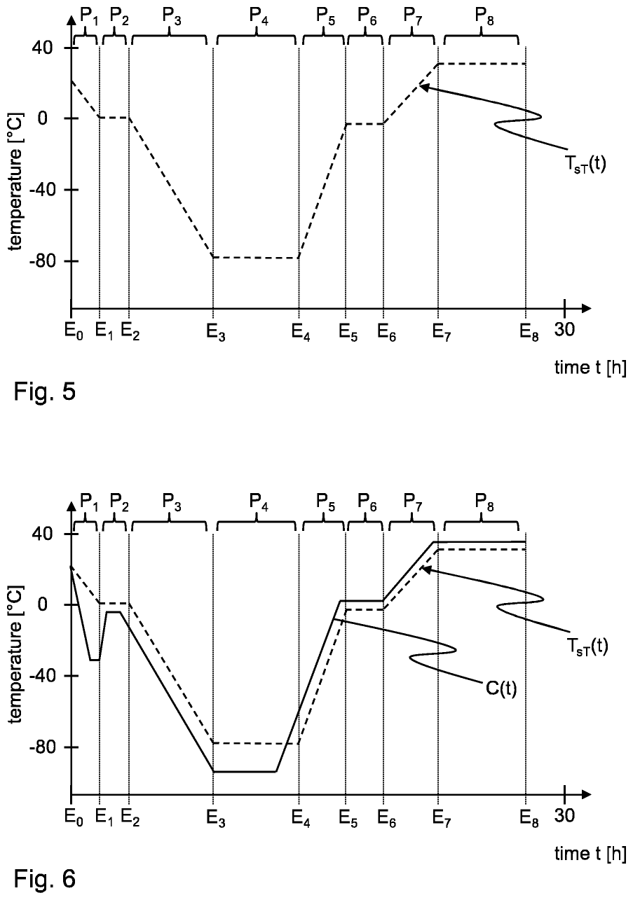

Optionally, in a next sub-step, the different phases P₁₋₈ of the target temperature course T_{T}(t) are adapted by applying linear regression. By applying linear regression, data points of the target temperature course associated to one of the different phases P₁₋₈ are represented as a linear function. The result of this sub-step is depicted in FIG. 5, in which each identified phase P₁₋₈ is represented and defined by a linear function resulting in a segmented target temperature course T_{sT}(t).

In step S3, based on the target temperature course T_{T}(t), in particular based on the segmented target temperature course T_{sT}(t), at least one preliminary control parameter variation C(t) is determined. In general, based on the control parameter variation C(t), the control unit 16 is configured to control operation of the heat exchanger unit 14. For doing so, the control unit 16 may use the control parameter variation C(t) as a control command or may derive control commands therefrom which are provided to the heat exchanger unit 14 as control signals via the data interface 34. Accordingly, based on the control parameter variation C(t), the heat exchanger unit 14 is configured to control heat transfer between the heat transfer component 18 and the aqueous formulation 12 during the operating cycle. In a further development, the step S3 of determining the control parameter variation may be further performed based on properties of the aqueous formulation, in particular based on physical or chemical properties of the aqueous formulation, e.g. as a function of the volume of the aqueous formulation, and/or based on properties of the container 24, in particular a type or volume of the container 24.

In the shown configuration, the at least one control parameter variation C(t) is indicative of a course of a temperature of the heat transfer component 18 during the operating cycle. More specifically, the control parameter variation C(t) is a variation of a temperature set point of the heat transfer medium directed through the heat transfer component 18. Specifically, the step of determining the control parameter variation is performed based on thermodynamic properties of the aqueous formulation, in particular based on the freezing and thawing point of the diluent (here: water). Specifically, this step is performed such that, during the freezing phase, the temperature set point of the heat transfer medium is set below the freezing point of the diluent (here: water) and, during the thawing phase, the temperature set point of the heat transfer medium is set above the thawing point of diluent (here: water). As can be gathered from the control parameter variation C(t) depicted in FIG. 6, this step is performed such that, during the freezing phase P2, the cooling phases P1 and P3 and the warming phases P5 and P7, the temperature set point of the heat transfer medium is varied. In other words, the control parameter variation C(t) defines a change of the temperature set point of the heat transfer medium during the cooling phases P1, P3, the freezing phase P2 and the warming phases P5 and P7. Alternatively, this step may be performed such that the control parameter variation C(t) defines a change of the temperature set point of the heat transfer medium during at least one of the first cooling phases P1, the freezing phase P2, the second cooling phase P3, the temperature holding phase P4, the first warming phase P5, the thawing phase P6 and the second warming phase P6.

Alternatively or additionally, a further control parameter variation may be determined which may be indicative of a flow rate of the heat transfer medium through the heat transfer component 18. Accordingly, the control parameter variation may define a change of the flow rate of the heat transfer medium during at least one of the first cooling phase P1, the freezing phase P2, the second cooling phase P3, the temperature holding phase P4, the first warming phase P5, the thawing phase P6 and the second warming phase P6, in particular during at least one of the first cooling phases P1, the freezing phase P2 and the second cooling phase P3.

FIG. 6 depicts an exemplary control parameter variation C(t) determined in step S3 which is illustrated as a graph in a diagram in which the graph representing the segmented target temperature course T_{sT}(t) is also shown. The exemplary control parameter variation C(t) depicts a course of a temperature set point of the heat transfer medium. The ordinate of the diagram depicts a temperature which corresponds to the value of the control variable, i.e. being indicative of a temperature of the heat transfer medium, in particular a set point of the temperature of the heat transfer medium. The abscissa of the diagram depicts the time during the operating cycle. As can be gathered in FIG. 6, determination of the control parameter variation C(t) is based on the types of the different phases P₁₋₈ of the segmented target temperature course T_{sT}(t) and a time period associated thereto. As can be gathered from FIG. 6, the temperature set point of the heat transfer medium is determined such that it lies below the freezing point of water during freezing phase P₂ and lies above the thawing point of water during thawing phase P₆. Specifically, during the thawing phase P₆, the temperature set point may be in the range of 2.5° C. to 20° C., preferably between 5° C. to 15° C. Further, the temperature set point of the heat transfer medium is determined such that it substantially lies below the target temperature course during cooling phases and above the target temperature course during warming phases. Further, in case the aqueous formulation 12 is to be held at a temperature, i.e. during a temperature holding phase, which is below an ambient temperature (i.e., about 21° C.), the temperature set point of the heat transfer medium is determined such that it substantially lies below the target temperature course, i.e. during the most time of the temperature holding phase. However, in case the aqueous formulation is to be held at a temperature, i.e. during a temperature holding phase, which is above an ambient temperature, the temperature set point of the heat transfer medium is determined such that it substantially lies above the target temperature course.

For example, for time periods associated to temperature variation phases, in particular a temperature decreasing or increasing phase, or temperature holding phases in the target temperature course T_{T}(t), the control unit 16 may define the control parameter variation C(t) in these phases based on the following equation:

$${\dot{T}}_{T \_ t} = A * ( {C}_{t} - {T}_{T \_ t} ) + B , \qquad ( 2 )$$

wherein \dot{T}_{T_t} refers to a temperature change rate in the target temperature course T_{T}(t) at time t; A and B refer to constants or coefficients, in particular aqueous formulation specific and heat exchanger specific constants or coefficient; Ct refers to a value of the control parameter at the time t; and T_{T_t} refers to a temperature value in the target temperature course T_{T}(t) at time t.

Alternatively or additionally, for carrying out step S3, the control unit 16 may use a reference control parameter variation which may be stored in a memory of the control unit 16 and which may refer to an operating cycle in which an aqueous formulation successively undergoes different phases corresponding to those phases identified in the segmented target temperature course T_{sT}(t). Alternatively or additionally, for determining the control parameter variation C(t), the control unit 16 may make use of a mathematical or computational model capable of predicting or calculating a course of temperature prevailing in the aqueous formulation 12 as a function of a control parameter variation. When using such a mathematical or computational model, the segmented target temperature course T_{sT}(t) may be used as an input, based on which the preliminary control parameter variation C(t) may be determined.

Further, the step (S3) of determining the control parameter variation C(t) may comprise a sub-step of identifying the freezing phase P₂ in the segmented target temperature course T_{sT}(t). Then, in a further sub-step, a descent is implemented in the control parameter variation C(t) during a time period of the operating cycle which precedes and/or overlaps with the time period associated with the freezing phase in the segmented target temperature course T_{sT}(t), as can be gathered from FIG. 6. The descent in the control parameter variation C(t) is designed so as to induce a temporal increase of the heat exchanger unit's cooling performance during the freezing phase P₂ in the operating cycle, in particular before and at the beginning of the freezing phase P₂.

Specifically, in the shown configuration, the control parameter variation C(t) is designed such that the temperature set point, during the first phase P₁, is decreased to a nucleation spike temperature which preferably is in the range between −25° C. to −35° C., for example −30° C. More specifically, in a first sub-phase of the first phase P₁, the temperature set point is decreased from an ambient temperature to the nucleation spike temperature. Then, the temperature set point is held at the nucleation spike temperature for a predetermined period of time, i.e. during a second sub-phase of the first Phase P₁. Specifically, the nucleation spike hold time may be in the range of 5% to 50% of the duration of the first phase P₁. Then, in a first sub-phase of the second phase P₂, the temperature set point is raised from the nucleation spike temperature to a freezing temperature. The freezing temperature may be in the range of −5° C. to −20° C., preferably in the range of −10° C. to −17.5° C., more preferably in the range of −12.5° C. to −17.5° C. Thereafter, in a second sub-phase of the second phase P₂, the temperature set point is kept at the freezing temperature. The duration of the first sub-phase of the second phase P₂ may be in the range of 1% to 20% of the duration of the second phase P₂. In a further development, the method may be provided such that step S4 is performed based on predefined temperature set points and timings, in particular based on at least one of a predetermined nucleation spike temperature, a predetermined nucleation spike hold time, a predetermined freezing temperature and a predetermined thawing temperature. Specifically, these set points or parameters may be set by an operator.

In a next step S4, the control unit 16 operates the heat exchanger unit 14 to perform a test run operating cycle. Specifically, this step comprises a sub-step S4.1 of operating the heat exchange unit 14 during the test run operating cycle based on the determined preliminary control parameter variation C(t). At the same time, a sub-step 4.2 of measuring a substance temperature course T_{M}(t) is carried out, wherein the substance temperature course T_{M}(t) is indicative of the course of the aqueous formulation's temperature during the test run operating cycle. This sub-step is performed by gathering measurement signal from the temperature sensor 32.

Then, in step S5, the measured temperature course T_{M}(t) is segmented into at least two distinct phases to obtain a segmented measured temperature course T_{sM}(t), wherein at least one phase refers to a change in physical state of the aqueous formulation 12. This step S5 may be performed corresponding to the above described step S2 of segmenting the target temperature course T_{T}(t). Specifically, step S5 is performed to identify a freezing phase, during which freezing of the aqueous formulation 12 has been performed, and a thawing phase, during which thawing of the aqueous formulation 12 has been performed. Further, step S5 may include a sub-steps of segmenting the measured temperature course T_{M}(t) into phases by identifying endpoints of the phases P₁₋₈; and a sub-step of adapting the phases P₁₋₈ of the measured temperature course T_{M}(t) by applying linear regression, thereby obtaining the segmented measured temperature course T_{sM}(t).

In a next step S6, the control unit 16 adapts the determined control parameter variation C(t) based on a comparison of the segmented measured temperature course T_{sM}(t) with the segmented target temperature course T_{sT}(t). For doing so, the control unit 16 may, at first, identify corresponding phases among the segmented measured temperature course T_{sM}(t) and the segmented target temperature course T_{sT}(t), i.e. phases of the same type which may occur at overlapping or at substantially the same or at close time periods during the operating cycle. Further, the control unit 16 may determine durations and/or an average temperature value of all identified phases. Then, based on a comparison of the determined durations and/or the determined average temperature values of the corresponding phases, the determined control parameter variation C(t) may be adapted.

Alternatively or additionally, the step S6 of adapting the control parameter variation C(t) may comprise the sub-steps of: identifying corresponding freezing phases P₂ or corresponding thawing phases P₆ among the segmented measured temperature course T_{sM}(t) and the segmented target temperature course T_{sT}(t); determining durations and average temperatures of the corresponding freezing phases P₂ or corresponding thawing phases P₆; and determining an average value of the control parameter variation C(t) in a time period corresponding to the freezing phase P₂ or thawing phase P₆ in the segmented measured temperature course T_{sM}(t); and adapting the control parameter variation C(t) as a function of the determined durations of the corresponding freezing phases or corresponding thawing phases, the determined average temperatures of the corresponding freezing phases or corresponding thawing phases and the determined average value of the control parameter variation.

Specifically, for doing so, in case the control unit 16 has identified corresponding changing physical state phases, in particular corresponding freezing phases or thawing phases in the segmented measured temperature course T_{sM}(t) and the segmented target temperature course T_{sT}(t), the control parameter variation C(t) may be adapted based on the following equation:

$${\overline{C}}_{S \_ P} = \frac{( {\overline{C}}_{M \_ P} - {\overline{T}}_{M \_ P} ) * {D}_{M \_ P} )}{{D}_{T \_ P}} + {\overline{T}}_{T \_ P} , \qquad ( 3 )$$

wherein
C̅_{S_P}, refers to a set value defining an average control parameter value to be set during a time period which refers to the corresponding phase in the target temperature course T_{T}(t) when adapting the control parameter variation C(t);
C̅_{M_p} refers to an average control parameter value of the control parameter variation C(t) during a time period which refers to the corresponding phase in the measured temperature course T_{S}(t);
T̅_{M_P} refers to an average temperature of the measured temperature course T_{M}(t) or the segmented measured temperature course T_{sM}(t) during a time period which refers to the corresponding phase in the measured temperature course T_{M}(t);
D_{M_P} refers to a duration of the corresponding phase in the measured temperature course T_{M}(t);
D_{T_P} refers to a duration of the corresponding phase in the target temperature course T_{M}(t); and
T̅_{T_P} refers to an average temperature of the target temperature course T_{T}(t) or the segmented target temperature course T_{sT}(t) during a time period which refers to the corresponding phase in the target temperature course T_{M}(t).

Further, for time periods associated to temperature variation phases, in particular a temperature decreasing or increasing phase, or temperature holding phases in the target temperature course T_{T}(t), the control unit 16 may adapt the control parameter variation C(t) in these phases based on the above described equation (2). Still further, for adapting the control parameter variation C(t) the control unit 16 may further make use of the mathematical or computational model used in step S3 for determining the control parameter variation C(t).

In a next optional step S7, the control unit 16 may determine a similarity parameter being indicative of a correlation or similarity between the target temperature course T_{T}(t) or the segmented target temperature course T_{sT}(t) with the measured temperature course T_{M}(t) or the segmented measured temperature course T_{sM}(t). Specifically, the similarity parameter may be calculated based on the following equation:

$$s = {\sum }_{t = {t}_{0}}^{{t}_{E}} \vert {T}_{T} ( t ) - {T}_{M} ( t ) \vert , \qquad ( 4 )$$

wherein s refers to the similarity parameter quantifying a similarity between the target temperature course T_{T}(t) and the measured temperature course T_{M}(t).

Then, in an optional step S8, the determined similarity parameters is compared with a threshold value S. If the similarity parameter s is below the threshold value S, the method proceeds to step S9 in which the control unit 16 allows to properly operate the heat exchanger unit 14, i.e. in accordance with the target temperature course T_{T}(t). If the similarity parameter s is equal to or greater than the threshold value S, the method goes back to step S4 to perform a further test run operating cycle, based on which the control parameter variation C(t) is further adapted. By this configuration, the method allows to iteratively adapt the control parameter variation C(t) until achieving satisfying results. In an alternative configuration, method step S6 may be performed after step S8, in particular if the similarity parameter s is equal to or greater than the threshold value S. According to one embodiment, method step S9 may be performed subsequent, in particular immediately after method step S6.

It will be obvious for a person skilled in the art that these embodiments and items only depict a plurality of possibilities for implementing the invention as defined in the independent claims. Hence, the embodiments shown here should not be understood to form a limitation of these features and configurations. Any possible combination and configuration of the described features can be chosen according to the scope of the invention as defined in the independent claims.

The present invention and embodiments thereof will be described again in the following clauses:
1. Method for operating a heat exchanger unit (14) for changing the physical state of matter of an aqueous formulation (12) by freezing or thawing,
   the aqueous formulation (12) being contained in a container and the heat exchanger unit acting on the aqueous formulation (12) in the container,
   such that a temperature of said aqueous formulation (12) during an operating cycle is regulated based on a target temperature course (T_{T}(t)), the method comprising the steps of:
(S2) segmenting the target temperature course (T_{T}(t)) into at least two distinct phases (P), wherein at least one phase (P₂, P₆) refers to a change in physical state of matter of the aqueous formulation (12);
(S3) based on the target temperature course (T_{T}(t)), determining a control parameter variation (C(t)) based on which operation of the heat exchanger unit (14) is to be controlled during an operating cycle;
(S4) performing a test run operating cycle comprising the sub-steps of:
   (S4.1) operating the heat exchange unit (14) during the test run operating cycle based on the determined control parameter variation (C(t)), and
   (S4.2) measuring a temperature course (T_{M}(t)) being indicative of the course of the aqueous formulation's temperature during the test run operating cycle;
(S5) segmenting the measured temperature course (T_{M}(t)) into at least two distinct phases, wherein at least one phase refers to a change in the physical state of the aqueous formulation (12);
(S6) adapting the determined control parameter variation (C(t)) based on a comparison of the segmented measured temperature course (T_{sM}(t)) with the segmented target temperature course (T_{sT}(t)); and
operating the heat exchanger unit (14) based on the adapted control parameter variation for changing the physical state by freezing or thawing the aqueous formulation (12).
2. Method according to clause 1, wherein said aqueous formulation (12) comprises water and optionally further at least one selected from the group consisting of a buffer, cell culture medium, and at least one or more drug substances.
3. Method according to clause 1 or 2, wherein the temperature of the aqueous formulation is measured by a sensor which is positioned in the container and preferably wherein the sensor is positioned in contact with the aqueous formulation in the container.
4. Method according to any one of clauses 1 to 3, wherein the heat exchanger unit (14) is configured to control heat transfer between a heat transfer component (18) and the aqueous formulation (12) during the operating cycle based on the determined control parameter variation (C(t)).
5. Method according to clause 4, wherein the at least one control parameter variation (C(t)) is indicative of a course of a temperature of the heat transfer component (18), in particular of a course of a temperature of a heat transfer medium flowing through the heat exchanger unit (14), during the operating cycle.
6. Method according to any one of clauses 1 to 5, wherein the control parameter variation (C(t)) defines a change of a control parameter of the heat exchanger unit (14) during the phase referring to the change in the physical state of the aqueous formulation (12).
7. Method according to any one of clauses 1 to 6, wherein the control parameter is indicative of a temperature of the heat transfer component (18), in particular is indicative of a temperature of the heat transfer medium flowing through the heat exchanger unit (16), or is indicative of a flow rate of the heat transfer medium.
8. Method according to any one of clauses 1 to 7, wherein the target temperature course (T_{T}(t)) corresponds to a temperature variation, the aqueous formulation (12) is subjected to upon being processed by a reference heat exchanger unit for changing a physical state of the aqueous formulation (12), wherein in particular the heat exchanger unit (14) is a scaled model of the reference heat exchanger unit.
9. Method according to any one of clauses 1 to 8, wherein at least one of the step (S2) of segmenting the target temperature course (T_{T}(t)) and the step (S5) of segmenting the measured temperature course (T_{M}(t)) is performed to identify at least one of a freezing phase (P₂), during which freezing of the aqueous formulation (12) is to be or has been performed, and a thawing phase (P₆), during which thawing of the aqueous formulation (12) is to be or has been performed.
10. Method according to any one of clauses 1 to 9, wherein at least one of the step (S2) of segmenting the target temperature course (T_{T}(t)) and the step (S5) of segmenting the measured temperature course (T_{M}(t)) includes at least one of:
   segmenting the target temperature course (T_{T}(t)) or the measured temperature course (T_{M}(t)) into phases by identifying endpoints of the phases; and
   adapting the phases of the target temperature course (T_{T}(t)) or the measured temperature course (T_{M}(t)) by applying linear regression.
11. Method according to any one of clauses 1 to 10, wherein the step (S3) of determining the control parameter variation (C(t)) is performed based on aqueous formulation's properties, in particular based on a volume of the aqueous formulation (12).
12. Method according to any one of clauses 1 to 11, wherein the step (S3) of determining the control parameter variation (C(t)) comprises:
   identifying a freezing phase (P₂) in the target temperature course (T_{T}(t)); and
   implementing a rise or descent in the control parameter variation (C(t)) in a time period of the operating cycle which precedes or overlaps with a time period associated to the freezing phase (P₂), wherein the rise or descent in the control parameter variation (C(t)) induces a temporal increase of the heat exchanger unit's cooling performance.
13. Method according to any one of clauses 1 to 12, wherein the step (S6) of adapting the control parameter variation C(t) comprises the sub-steps of:
   identifying corresponding phases among the segmented measured temperature course (T_{sM}(t)) and the segmented target temperature course (T_{sT}(t));
   determining durations of the corresponding phases and/or determining average temperatures of the correspond phases; and
   based on comparison of the determined durations and/or average temperatures of the corresponding phases, adapting the control parameter variation (C(t)).
14. Method according to any one of clause 1 to clause 13, wherein the step (S6) of adapting the control parameter variation (C(t)) comprises the sub-steps of:
   identifying corresponding freezing phases or corresponding thawing phases among the segmented measured temperature course (T_{sM}(t)) and the segmented target temperature course (T_{sT}(t));
   determining durations and average temperatures of the corresponding freezing phases or the corresponding thawing phases; and
   determining an average value of the control parameter variation (C(t)) in a time period corresponding to the freezing phase or thawing phase in the segmented measured temperature course (T_{sT}(t)); and
   adapting the control parameter variation (C(t)) as a function of the determined durations of the corresponding freezing phases or corresponding thawing phases, the determined average temperatures of the corresponding freezing phases or the corresponding thawing phases, and the determined average value of the control parameter variation C(t).
15. A data processing system for controlling a heat exchanger unit (14) configured to change the physical state of matter of an aqueous formulation (12) by freezing or thawing, wherein the aqueous formulation (12) is contained in a container and the heat exchanger unit acts on the aqueous formulation (12) in the container, such that a temperature of said aqueous formulation (12) during an operating cycle is regulated based on a target temperature course (T_{T}(t)), and further wherein the data processing system comprises a central processing unit, memory, and software stored in memory which, when executed by the central processing unit, performs the steps of:
   (S2) segmenting the target temperature course (T_{T}(t)) into at least two distinct phases (P), wherein at least one phase (P₂, P₆) refers to a change in physical state of matter of the aqueous formulation (12);
   (S3) based on the target temperature course (T_{T}(t)), determining a control parameter variation (C(t)) based on which operation of the heat exchanger unit (14) is to be controlled during an operating cycle;
   (S4) performing a test run operating cycle comprising the sub-steps of:
(S4.1) operating the heat exchange unit (14) during the test run operating cycle based on the determined control parameter variation (C(t)), and
(S4.2) measuring a temperature course (T_{M}(t)) being indicative of the course of the aqueous formulation's temperature during the test run operating cycle;
   (S5) segmenting the measured temperature course (T_{M}(t)) into at least two distinct phases, wherein at least one phase refers to a change in the physical state of the aqueous formulation (12);
   (S6) adapting the determined control parameter variation (C(t)) based on a comparison of the segmented measured temperature course (T_{sM}(t)) with the segmented target temperature course (T_{sT}(t)); and
   operating the heat exchanger unit (14) based on the adapted control parameter variation for changing the physical state by freezing or thawing the aqueous formulation (12).
16. A computer program for controlling a heat exchanger unit (14) configured to change the physical state of matter of an aqueous formulation (12) by freezing or thawing, wherein the aqueous formulation (12) is contained in a container and the heat exchanger unit acts on the aqueous formulation (12) in the container, such that a temperature of said aqueous formulation (12) during an operating cycle is regulated based on a target temperature course (T_{T}(t)), wherein the computer program comprises instructions, which when the computer program is executed by a computer, causes the computer to carry out the steps of:
   (S2) segmenting the target temperature course (T_{T}(t)) into at least two distinct phases (P), wherein at least one phase (P₂, P₆) refers to a change in physical state of matter of the aqueous formulation (12);
   (S3) based on the target temperature course (T_{T}(t)), determining a control parameter variation (C(t)) based on which operation of the heat exchanger unit (14) is to be controlled during an operating cycle;
   (S4) performing a test run operating cycle comprising the sub-steps of:
(S4.1) operating the heat exchange unit (14) during the test run operating cycle based on the determined control parameter variation (C(t)), and
(S4.2) measuring a temperature course (T_{M}(t)) being indicative of the course of the aqueous formulation's temperature during the test run operating cycle;
   (S5) segmenting the measured temperature course (T_{M}(t)) into at least two distinct phases, wherein at least one phase refers to a change in the physical state of the aqueous formulation (12);
   (S6) adapting the determined control parameter variation (C(t)) based on a comparison of the segmented measured temperature course (T_{sM}(t)) with the segmented target temperature course (T_{sT}(t)); and
   operating the heat exchanger unit (14) based on the adapted control parameter variation for changing the physical state by freezing or thawing the aqueous formulation (12).
17 A computer program according to clause 16 wherein said computer program is stored on a computer-readable medium
18. A computer-readable medium having stored thereon a computer program for controlling a heat exchanger unit (14) configured to change the physical state of matter of an aqueous formulation (12) by freezing or thawing, wherein the aqueous formulation (12) is contained in a container and the heat exchanger unit acts on the aqueous formulation (12) in the container, such that a temperature of said aqueous formulation (12) during an operating cycle is regulated based on a target temperature course (T_{T}(t)), wherein the computer program comprises instructions, which when the computer program is executed by a computer, causes the computer to carry out the steps of:
   (S2) segmenting the target temperature course (T_{T}(t)) into at least two distinct phases (P), wherein at least one phase (P₂, P₆) refers to a change in physical state of matter of the aqueous formulation (12);
   (S3) based on the target temperature course (T_{T}(t)), determining a control parameter variation (C(t)) based on which operation of the heat exchanger unit (14) is to be controlled during an operating cycle;
   (S4) performing a test run operating cycle comprising the sub-steps of:
(S4.1) operating the heat exchange unit (14) during the test run operating cycle based on the determined control parameter variation (C(t)), and
(S4.2) measuring a temperature course (T_{M}(t)) being indicative of the course of the aqueous formulation's temperature during the test run operating cycle;
   (S5) segmenting the measured temperature course (T_{M}(t)) into at least two distinct phases, wherein at least one phase refers to a change in the physical state of the aqueous formulation (12);
   (S6) adapting the determined control parameter variation (C(t)) based on a comparison of the segmented measured temperature course (T_{sM}(t)) with the segmented target temperature course (T_{sT}(t)); and
   operating the heat exchanger unit (14) based on the adapted control parameter variation for changing the physical state by freezing or thawing the aqueous formulation (12).
19. System (10) comprising a heat exchanger unit (14) and a control unit (16) for operating the heat exchanger unit (14) for changing the physical state of an aqueous formulation (12) such that a temperature of the aqueous formulation (12) is regulated during an operating cycle based on a target temperature course (T_{T}(t)), wherein the control unit (16) is configured to carry out the steps of:
   (S2) segmenting the target temperature course (T_{T}(t)) into at least two distinct phases (P), wherein at least one phase (P₂, P₆) refers to a change in physical state of matter of the aqueous formulation (12);
   (S3) based on the target temperature course (T_{T}(t)), determining a control parameter variation (C(t)) based on which operation of the heat exchanger unit (14) is to be controlled during an operating cycle;
   (S4) performing a test run operating cycle comprising the sub-steps of:
      (S4.1) operating the heat exchange unit (14) during the test run operating cycle based on the determined control parameter variation (C(t)), and
      (S4.2) measuring a temperature course (T_{M}(t)) being indicative of the course of the aqueous formulation's temperature during the test run operating cycle;
   (S5) segmenting the measured temperature course (T_{M}(t)) into at least two distinct phases, wherein at least one phase refers to a change in the physical state of the aqueous formulation (12);
   (S6) adapting the determined control parameter variation (C(t)) based on a comparison of the segmented measured temperature course (T_{sM}(t)) with the segmented target temperature course (T_{sT}(t)); and
   operating the heat exchanger unit (14) based on the adapted control parameter variation for changing the physical state by freezing or thawing the aqueous formulation (12).

### LIST OF REFERENCE NUMERALS

10 heat exchanger system
12 aqueous formulation
14 heat exchanger unit
16 control unit
16A CPU
16B memory
18 heat transfer component
20 heat transfer plates
22 receiving space
24 container
26 temperature control unit
28 feed line
30 discharge line
32 temperature sensor
34 data interface
C(t) control parameter variation
E phase end point
P phase
Q heat flow
t point in time
s similarity measure
S threshold
T_{T}(t) target temperature course
T_{sT}(t) segmented target temperature course
T_{M}(t) measured temperature course
T_{sM}(t) segmented measures temperature course

1. Method for operating a heat exchanger unit (14) for changing the physical state of matter of an aqueous formulation (12) by freezing or thawing,
the aqueous formulation (12) being contained in a container and the heat exchanger unit acting on the aqueous formulation (12) in the container,
such that a temperature of said aqueous formulation (12) during an operating cycle is regulated based on a target temperature course (T_{T}(t)), the method comprising the steps of:
(S2) segmenting the target temperature course (T_{T}(t)) into at least two distinct phases (P), wherein at least one phase (P₂, P₆) refers to a change in physical state of matter of the aqueous formulation (12);
(S3) based on the target temperature course (T_{T}(t)), determining a control parameter variation (C(t)) based on which operation of the heat exchanger unit (14) is to be controlled during an operating cycle;
(S4) performing a test run operating cycle comprising the sub-steps of:
(S4.1) operating the heat exchange unit (14) during the test run operating cycle based on the determined control parameter variation (C(t)), and
(S4.2) measuring a temperature course (T_{M}(t)) being indicative of the course of the aqueous formulation's temperature during the test run operating cycle;
(S5) segmenting the measured temperature course (T_{M}(t)) into at least two distinct phases, wherein at least one phase refers to a change in the physical state of the aqueous formulation (12);
(S6) adapting the determined control parameter variation (C(t)) based on a comparison of the segmented measured temperature course (T_{sM}(t)) with the segmented target temperature course (T_{sT}(t)); and
operating the heat exchanger unit (14) based on the adapted control parameter variation for changing the physical state by freezing or thawing the aqueous formulation (12).

2. Method according to claim 1, wherein said aqueous formulation (12) comprises water and optionally further a buffer, cell culture medium, and/or one or more drug substances.

3. Method according to claim 1, wherein the temperature of the aqueous formulation is measured by a sensor which is positioned in the aqueous formulation, in particular in the container.

4. Method according to claim 1, wherein the heat exchanger unit (14) is configured to control heat transfer between a heat transfer component (18) and the aqueous formulation (12) during the operating cycle based on the determined control parameter variation (C(t)).

5. Method according to claim 4, wherein the at least one control parameter variation (C(t)) is indicative of a course of a temperature of the heat transfer component (18), in particular of a course of a temperature of a heat transfer medium flowing through the heat exchanger unit (14), during the operating cycle.

6. Method according to claim 1, wherein the control parameter variation (C(t)) defines a change of a control parameter of the heat exchanger unit (14) during the phase referring to the change in the physical state of the aqueous formulation (12).

7. Method according to claim 1, wherein the control parameter is indicative of a temperature of the heat transfer component (18), in particular is indicative of a temperature of the heat transfer medium flowing through the heat exchanger unit (16), or is indicative of a flow rate of the heat transfer medium.

8. Method according to claim 1, wherein the target temperature course (T_{T}(t)) corresponds to a temperature variation, the aqueous formulation (12) is subjected to upon being processed by a reference heat exchanger unit for changing a physical state of the aqueous formulation (12), wherein in particular the heat exchanger unit (14) is a scaled model of the reference heat exchanger unit.

9. Method according to claim 1, wherein at least one of the step (S2) of segmenting the target temperature course (T_{T}(t)) and the step (S5) of segmenting the measured temperature course (T_{M}(t)) is performed to identify at least one of a freezing phase (P₂), during which freezing of the aqueous formulation (12) is to be or has been performed, and a thawing phase (P₆), during which thawing of the aqueous formulation (12) is to be or has been performed.

10. Method according to claim 1, wherein at least one of the step (S2) of segmenting the target temperature course (T_{T}(t)) and the step (S5) of segmenting the measured temperature course (T_{M}(t)) includes at least one of:
segmenting the target temperature course (T_{T}(t)) or the measured temperature course (T_{M}(t)) into phases by identifying endpoints of the phases; and
adapting the phases of the target temperature course (T_{T}(t)) or the measured temperature course (T_{M}(t)) by applying linear regression.

11. Method according to claim 1, wherein the step (S3) of determining the control parameter variation (C(t)) is performed based on aqueous formulation's properties, in particular based on a volume of the aqueous formulation (12).

12. Method according to claim 1, wherein the step (S3) of determining the control parameter variation (C(t)) comprises:
identifying a freezing phase (P₂) in the target temperature course (T_{T}(t)); and
implementing a rise or descent in the control parameter variation (C(t)) in a time period of the operating cycle which precedes or overlaps with a time period associated to the freezing phase (P₂), wherein the rise or descent in the control parameter variation (C(t)) induces a temporal increase of the heat exchanger unit's cooling performance.

13. Method according to claim 1, wherein the step (S6) of adapting the control parameter variation C(t) comprises the sub-steps of:
identifying corresponding phases among the segmented measured temperature course (T_{sM}(t)) and the segmented target temperature course (T_{sT}(t));
determining durations of the corresponding phases and/or determining average temperatures of the correspond phases; and
based on comparison of the determined durations and/or average temperatures of the corresponding phases, adapting the control parameter variation (C(t)).

14. Method according to claim 1, wherein the step (S6) of adapting the control parameter variation (C(t)) comprises the sub-steps of:
identifying corresponding freezing phases or corresponding thawing phases among the segmented measured temperature course (T_{sM}(t)) and the segmented target temperature course (T_{sT}(t));
determining durations and average temperatures of the corresponding freezing phases or the corresponding thawing phases; and
determining an average value of the control parameter variation (C(t)) in a time period corresponding to the freezing phase or thawing phase in the segmented measured temperature course (T_{sT}(t)); and
adapting the control parameter variation (C(t)) as a function of the determined durations of the corresponding freezing phases or corresponding thawing phases, the determined average temperatures of the corresponding freezing phases or the corresponding thawing phases, and the determined average value of the control parameter variation C(t).

15. A data processing system comprising means for carrying out the steps of the method of claim 1.

16. A computer program comprising instructions which, when the program is executed by a computer, cause the computer to carry out the steps of the method of claim 1.

17. A computer-readable medium having stored thereon the computer program as defined in claim 16.

18. System (10) comprising a heat exchanger unit (14) and a control unit (16) for operating the heat exchanger unit (14) for changing the physical state of an aqueous formulation (12) such that a temperature of the aqueous formulation (12) is regulated during an operating cycle based on a target temperature course (T_{T}(t)), wherein the control unit (16) is configured to carry out the steps of the method of claim 1.
